(19) United States
(12) Patent Application Publication
(10) Pub. No.: US 2007/0269497 A1
(43) Pub. Date: Nov. 22, 2007

(54) Chemical stick with soluble stabilizer

(76) Inventor: Lee Alves, Hobbs, NM (US)

Correspondence Address: LITMAN LAW OFFICES, LTD., P.O. BOX 15035, CRYSTAL CITY STATION, ARLINGTON, VA, 22215, US

(21) Appl. No.: 11/434,746

(22) Filed: May 17, 2006

(51) Int. Cl. A61K 9/70

(52) U.S. Cl. 424/443

(57) ABSTRACT

The chemical stick with soluble stabilizer eliminates or greatly reduces the problem of fractured chemical treatment sticks in wells, where such sticks occasionally break into relatively short lengths and jam crosswise within the well pipe. The chemical stick includes one or more water soluble stabilizer elements that extend substantially the length of the stick, and which add to the physical strength of the stick to greatly reduce breakage. The stabilizer or reinforcement element(s) are also sufficiently flexible to avoid fracturing if the remainder of the body of the stick is cracked or fractured. The reinforcement element(s) maintain the integrity of the stick, and prevent its separation into separate fragments. Polyvinyl alcohol (PVA) is a suitable stick stabilizer, as polyvinyl alcohol is water soluble in order to dissolve in the well and does not adversely affect the detergent or surfactant in the well treatment stick.

### BACKGROUND OF THE INVENTION

1. Field of the Invention

The present invention relates generally to chemical additives used in the treatment of wells. More specifically, the present invention relates to a chemical stick with soluble stabilizer that is formed of detergent of other surfactant into a stick, commonly known as a “soap stick,” used to remove water from gas wells and that has a stabilizer incorporated into the chemical stick to prevent the stick from fracturing into fragments that might jam the well pipe.

2. Description of the Related Art

Gas production wells are often prone to contamination by water encroaching the underground gas field as gas pressure is reduced during production. Oftentimes, the flow of gas is insufficient to remove the water from the well, and the hydrostatic pressure of the water in the well can become sufficiently high that the pressure may reduce or even stop the flow of gas from the well.

A solution to the above problem has been developed in which sticks (or other form) of detergent or surfactant are dropped into the well pipe. The detergent or surfactant inserts, commonly known as “soap sticks,” cause the water in the well to foam, with the relatively low pressure foam being carried from the well along with the gas as the gas flows from the well. This technique has been found to increase gas production significantly in many otherwise unproductive wells.

A problem with such soap sticks is that the sticks tend to be somewhat fragile and susceptible to fracture or other damage. This can also be true of other types of chemical sticks used to treat gas wells and other types of wells. Generally, the chemicals used are formed into relatively long and narrow sticks, e.g., on the order of about an inch or so in diameter by about fifteen inches long (other dimensions may be used as well). This allows the sticks to drop freely down through a well pipe without jamming in the pipe, assuming the stick maintains its integrity.

However, the materials from which such sticks are formed result in the sticks being somewhat fragile due to their lengths and relatively narrow diameters, and such sticks are prone to fracturing during handling. When such a stick fractures into two or more shorter lengths, a stick fraction having a length slightly longer than the internal diameter of the well pipe has a good likelihood of turning crosswise in the well pipe and jamming in the pipe before reaching the bottom of the well. When this happens, subsequent sticks dropped into the well will be stopped by the jammed portion and will likely wedge the jammed portion even more tightly into place within the well pipe as they impact it at the end of their fall. This may result in whole sticks breaking up when they impact the jammed fragment, resulting in an even more complex blockage. This requires that the sticks (both whole, if any, and fragmented) be “fished” from the well, a process that is tedious and time consuming.

Broken sticks can also result in the jamming of other equipment and machinery, e.g., automated stick feeders or dispensers in use at some gas wells. In cases where the sticks are damaged in shipping or before placement into the well or dispensing machinery, they must be discarded, as they cannot be reliably transferred through the machinery and/or into the well.

Accordingly, there have been some attempts to rectify this potential problem, such as forming the chemical sticks with homogeneous additives that reduce their brittleness and fragility. However, the mixing of additives that affect the physical properties of the chemical stick also affects the chemistry of the stick and may reduce its effectiveness and/or result in undesired reactions.

A number of non-homogeneous inserts have been developed for use within cakes of bath soap, for the purpose of allowing the entire volume of soap to be used by using the insert for handling the remaining soap bar as it dissipates during use. An example of such is found in Japanese Patent No. 01-123,900, published on May 16, 1989. According to the drawings and English abstract, a core formed of expanded polystyrene, metal, wood, or other water insoluble material is provided, with the soap being formed around the core.

Another example of such a soap cake insert is found in Japanese Patent No. 11-116,998 published on Apr. 27, 1999. The '998 patent discloses (according to the drawings and English abstract) a cake of bath soap having a water insoluble insert therein. The insert may be buoyant to enable the soap to float, and may comprise an open or closed cell foam material.

None of the above inventions and patents, taken either singly or in combination, is seen to describe the instant invention as claimed. Thus, a chemical stick with a soluble stabilizer solving the aforementioned problems is desired.

### SUMMARY OF THE INVENTION

The chemical stick with soluble stabilizer eliminates, or at least greatly reduces, the problem of fractured chemical treatment sticks in wells and other equipment, particularly where detergent/surfactant or “soap sticks” are used to reduce water accumulation in gas wells. The chemical stick includes one or more water-soluble reinforcement elements, which extend substantially the length of the stick, and which add to the physical strength of the stick to greatly reduce breakage. The stabilizer or reinforcement element(s) also provide(s) sufficient flexibility to avoid fracturing if the remainder of the body of the stick is cracked or fractured. The reinforcement element(s) maintain(s) the integrity of the stick, and prevent its separation into separate fragments.

The soluble stabilizer material is preferably a polyvinyl alcohol (PVA) material, which is a flexible solid, soluble in water, and does not produce any undesired physical or chemical reactions when used as the reinforcement for a soap stick to reduce water flooding in a gas well. Polyvinyl alcohol is also compatible with numerous other chemicals that might be used in well treatment sticks. However, other stabilizing and/or reinforcing materials providing the required properties may be used as well.

These and other features of the present invention will become readily apparent upon further review of the following specification and drawings.

### BRIEF DESCRIPTION OF THE DRAWINGS

FIG. 1A is an elevation view of an unbroken chemical stick with soluble stabilizer according to the present invention, showing a single stabilizer element therein in broken lines.

FIG. 1B is an elevation view of a chemical stick according to the present invention similar to the stick of FIG. 1A, but showing the continued integrity of the stick after fracturing.

FIG. 2 is an enlarged schematic end elevation view of an alternative embodiment of a chemical stick according to the present invention, showing a series of different patterns or configurations for the soluble stabilizer formed therein.

FIG. 3 is a prior art side elevation view in section of a well pipe with a section of broken chemical stick jammed therein, illustrating the problem solved by the chemical stick with soluble stabilizer according to the present invention.

Similar reference characters denote corresponding features consistently throughout the attached drawings.

### DETAILED DESCRIPTION OF THE PREFERRED EMBODIMENTS

The present invention is a chemical stick with soluble stabilizer that includes a water soluble stabilizer therein, primarily for the purpose of treating wells, and most particularly for the treatment of gas wells. The chemical stick or well treatment stick may comprise any of a number of different chemical compositions, but such sticks are most commonly formed of a water soluble detergent or surfactant material in order to cause the water in the well to foam. The foam and gas mixture is then easily carried to the surface by the gas pressure in the well.

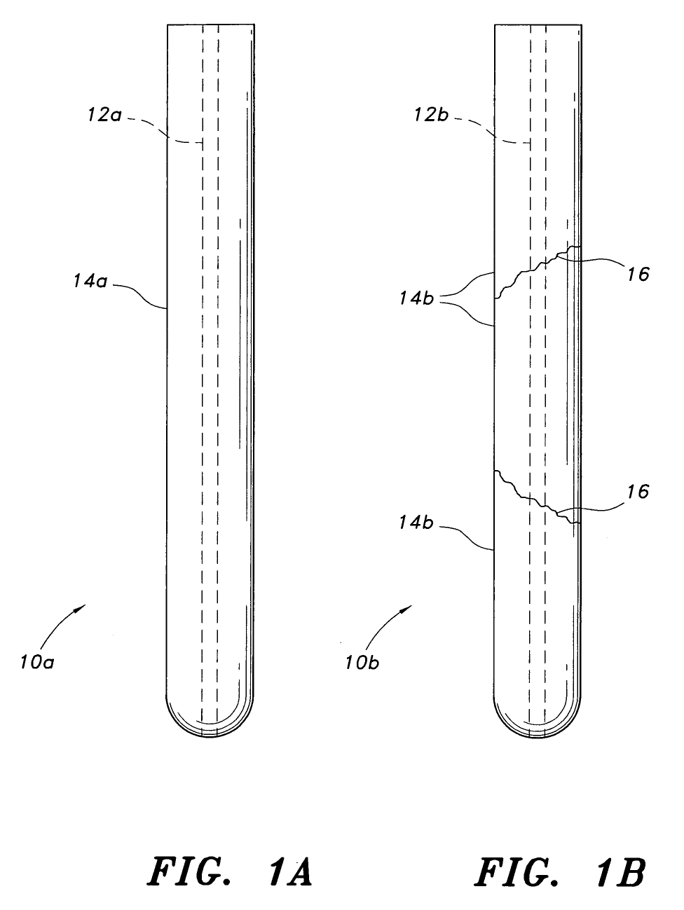

FIGS. 1A and 1B illustrate side elevation views of a pair of exemplary chemical treatment sticks of the present invention, respectively 10a and 10b. The exemplary chemical treatment stick 10a is formed as an elongate, narrow rod of water soluble well treatment material configured to fit easily through the down pipe of a gas well or the like. Such chemical treatment sticks are generally relatively fragile, and conventional chemical treatment sticks easily separate into fragments when cracked or fractured. Such fragments can easily jam within the well pipe, thus necessitating a costly and tedious effort to “fish” them from the pipe.

The chemical treatment stick 10a provides considerably greater resistance to this frangibility by means of at least one flexible, water soluble stabilizer and reinforcement element 12a formed therein. The stabilizer and reinforcement element 12a may be formed of any flexible, water soluble material. Polyvinyl alcohol (PVA) has been found to be suitable for this purpose, as in its substantially dehydrated state it forms a flexible plastic material with reasonably good strength. The surrounding chemical treatment material 14a, e.g., detergent/surfactant, etc., may be cast or molded in place about the stabilizer and reinforcement element 12a so that the stabilizer 12a forms a central core for the stick 10a.

The chemical treatment material 14a (e.g., detergent/surfactant, etc.) adheres to the flexible stabilizer element 12a, once the material 14a has cured or hardened. In the event that the relatively brittle or frangible chemical treatment material 14a becomes cracked, as shown in the material 14b of the otherwise identical chemical treatment stick 10b of FIG. 1B, the flexible nature of the stabilizer and reinforcement element 12b continues to hold the material 14b together in a single, cohesive unit, rather than allowing the pieces to separate along their cracks 16. The stabilizer and reinforcement element 12a, 12b also adds some additional strength to the chemical stick 10a, 10b, providing some additional resistance against breakage in the first place.

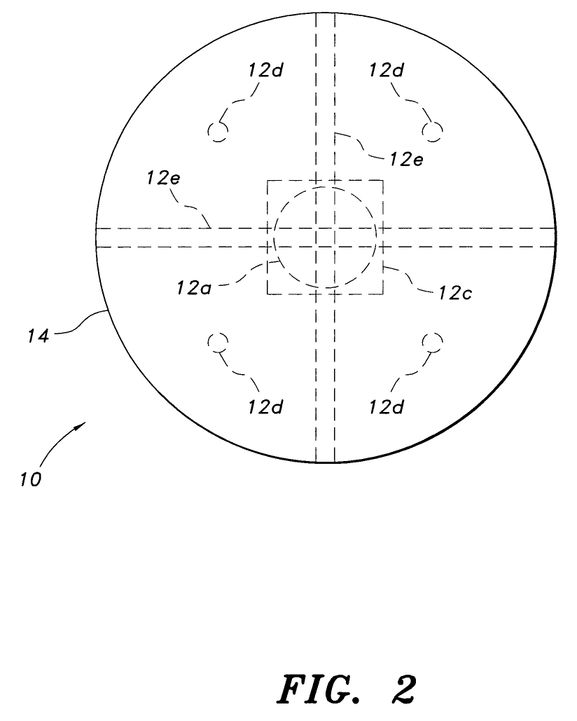

The exact placement and configuration of the stabilizer and reinforcement element within the chemical treatment material is not particularly critical, so long as it performs the tasks of strengthening the chemical treatment material and preventing the material from separating into smaller fragments if the stick becomes cracked or fractured. FIG. 2 provides a series of exemplary patterns for stabilizer and reinforcement elements in an enlarged end view of a chemical treatment stick 10. The central reinforcement element 12a will be seen to have a circular cross section substantially concentric within the stick or rod 10, and is essentially the same element configuration as the elements 12a and 12b shown in the chemical sticks 10a and 10b of FIGS. 1A and 1B.

However, the stabilizer and reinforcement element may take on any of innumerable cross-sectional shapes. Another example of such a reinforcement element 12c is indicated by the square cross-sectional configuration in FIG. 2. Other non-square regular or irregular polygonal cross-sectional shapes may be provided. Alternatively, the stabilizer and reinforcement element may comprise a plurality of separate elements 12d spaced outwardly from the center of the treatment material 14 in a symmetrical or an asymmetrical array, if so desired.

Another alternative, shown in FIG. 2, is the relatively thin, flat sheet stabilizer configuration 12e, with the sheet essentially spanning the diameter of the rod or stick 10. Two such stabilizer and reinforcement sheets 12e may be placed within the treatment material 14, as shown, or a single sheet 12e may be sufficient. It will be seen that additional such sheets may be used to strengthen and reinforce the rod or stick 10, and/or one or more such sheets 12e may be incorporated with other stabilizer and reinforcement elements 12a, 12b, 12c, and/or 12d.

The specific configuration of the stabilizer and reinforcement element(s) is not particularly critical, so long as the stabilizer accomplishes its purpose of strengthening the rod or stick and preventing separation of fractional stick components in the event the stick is broken. It should be noted that while the various exemplary reinforcement patterns are shown in broken lines in the end view of FIG. 2, the reinforcements 12a, 12b, 12c, etc. may continue completely through the length of the chemical stick, or may terminate somewhat short of either or both ends, so long as the stabilizer extends through a sufficient portion of the length of the stick to provide the required reinforcement.

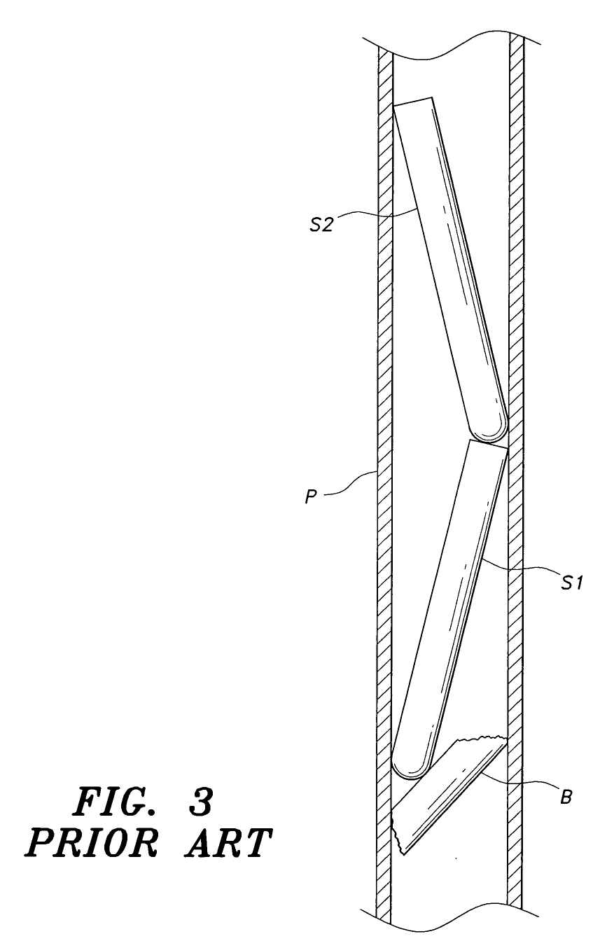

FIG. 3 provides an elevation view in section of an exemplary well pipe P, and illustrates the problem that may occur when a chemical treatment stick breaks at some point during its passage into the well. In FIG. 3, a section of broken well treatment stick B slightly longer than the inside diameter of the well pipe P has become jammed or lodged across the interior of the well pipe. This leads to the problem of a subsequent treatment stick S1 falling through the pipe P until it encounters the jammed section of broken stick B, whereupon the stick S1 is stopped by the broken stick B. Another following stick S2 then encounters the stick S1, or perhaps falls directly onto the broken stick section B, whereupon the stick S2 becomes jammed. One problem is that the well operator will not likely become immediately aware of the jammed stick problem, and will only recognize the problem after some time has passed and additional sticks have become jammed in the well pipe P. At this point, it is a difficult, tedious, and time-consuming procedure to remove what may be a considerable number of broken and/or jammed sticks B, S1, S2, etc., from the well pipe P.

Accordingly, the chemical stick with soluble stabilizer provides a much needed improvement over conventional chemical sticks of the prior art, with their relative fragility and tendency to break. The stabilizer and reinforcement elements within the sticks maintain the integrity of each stick, assuring that the sticks will fall through the well pipe without breaking up and having smaller lengths or sections jam within the pipe. The sticks behave conventionally once they reach the water at the bottom of the well, dissolving within the water to release their chemical action, e.g., acting as a surfactant to produce a foam to allow the water to be carried to the surface with the release of gas from the well, etc. When the outer chemical treatment material dissolves sufficiently to expose the stabilizer and reinforcement elements within the sticks, those elements (polyvinyl alcohol, or other suitable water soluble material) also dissolve. Thus, the chemical stick with soluble stabilizer has no insoluble elements to be left behind in the well to cause potential problems.

While the problems occurring due to a broken stick within the well have been noted herein, it will be recognized that the breakage of a chemical stick at virtually any point in the process can create time consuming problems. For example, automated machinery has been developed to dispense such chemical treatment sticks into gas wells, without the need or intervention of a human operator. If a conventional stick breaks and becomes jammed within the dispensing machinery, an operator will need to take the time to remedy the situation. The operator may not become aware of the problem or be able to travel to the site to resolve the problem for some time if the machine is located at a remote site. Accordingly, the use of the chemical stick with soluble stabilizer will greatly reduce the problem of broken well treatment sticks in wells, dispensing machinery, and other areas where such broken sticks may create a problem.

It is to be understood that the present invention is not limited to the embodiments described above, but encompasses any and all embodiments within the scope of the following claims.

I claim:

1. A chemical stick with soluble stabilizer, comprising:
an elongate, narrow rod of water soluble well treatment chemical; and
at least one flexible, water soluble stabilizer element disposed within the rod, the well treatment chemical adhering to the stabilizer element so that the stabilizer element maintains the rod's original shape and precludes separation of fractured rod components from one another when the rod becomes fractured.

2. The chemical stick with soluble stabilizer according to claim 1, wherein said rod of water soluble well treatment chemical comprises a surfactant stick.

3. The chemical stick with soluble stabilizer according to claim 1, wherein said at least one water soluble stabilizer comprises polyvinyl alcohol.

4. The chemical stick with soluble stabilizer according to claim 1, wherein said at least one water soluble stabilizer element comprises a homogenous elongate element disposed substantially concentrically within said rod.

5. The chemical stick with soluble stabilizer according to claim 1, wherein said at least one stabilizer element comprises a plurality of stabilizer elements.

6. The chemical stick with soluble stabilizer according to claim 1, wherein said at least one stabilizer element has a circular cross section.

7. The chemical stick with soluble stabilizer according to claim 1, wherein said at least one stabilizer element has a polygonal cross section.

8. The chemical stick with soluble stabilizer according to claim 1, wherein said at least one stabilizer element comprises at least one thin, flat sheet substantially spanning said rod.

9. A chemical stick with soluble stabilizer, comprising:
an elongate, narrow rod of water soluble well treatment surfactant material; and
at least one flexible, water soluble stabilizer element disposed within the rod, the surfactant material adhering to the stabilizer element so that the stabilizer element maintains the rod's original shape and precludes separation of fractured rod components from one another when the rod becomes fractured.

10. The chemical stick with soluble stabilizer according to claim 9, wherein said at least one water soluble stabilizer element comprises polyvinyl alcohol.

11. The chemical stick with soluble stabilizer according to claim 9, wherein said at least one water soluble stabilizer element comprises a homogenous elongate element disposed substantially concentrically within said rod.

12. The chemical stick with soluble stabilizer according to claim 9, wherein said at least one stabilizer element comprises a plurality of stabilizer elements.

13. The chemical stick with soluble stabilizer according to claim 9, wherein said at least one stabilizer element has a shape selected from the group consisting of circular and polygonal cross sections.

14. The chemical stick with soluble stabilizer according to claim 9, wherein said at least one stabilizer element comprises at least one thin, flat sheet substantially spanning said rod.

15. A chemical stick with soluble stabilizer, comprising:
an elongate, narrow rod of water soluble well treatment chemical; and
at least one flexible, water soluble stabilizer element formed of polyvinyl alcohol disposed within the rod, the well treatment chemical adhering to the stabilizer element so that the stabilizer element maintains the rod's original shape and precludes separation of fractured rod components from one another when the rod becomes fractured.

16. The chemical stick with soluble stabilizer according to claim 15, wherein said rod of water soluble well treatment chemical comprises a surfactant stick.

17. The chemical stick with soluble stabilizer according to claim 15, wherein said at least one water soluble stabilizer element comprises a homogenous elongate element disposed substantially concentrically within said rod.

18. The chemical stick with soluble stabilizer according to claim 15, wherein said at least one stabilizer element comprises a plurality of stabilizer elements.

19. The chemical stick with soluble stabilizer according to claim 15, wherein said at least one stabilizer element has a shape selected from the group consisting of circular and polygonal cross sections.

20. The chemical stick with soluble stabilizer according to claim 15, wherein said at least one stabilizer element comprises at least one thin, flat sheet substantially spanning said rod.
